(19) United States
(12) Patent Application Publication
(10) Pub. No.: US 2011/0093068 A1
(43) Pub. Date: Apr. 21, 2011

(54) CAPSULE CAPTURE SNARE

(75) Inventor: Theodore P. WERBLIN, Princeton, WV (US)

(73) Assignee: WERBLIN RESEARCH & DEVELOPMENT CORP., Princeton, WV (US)

(21) Appl. No.: 12/908,335

(22) Filed: Oct. 20, 2010

Related U.S. Application Data
(60) Provisional application No. 61/253,636, filed on Oct. 21, 2009.

(51) Int. Cl. A61F 2/16

(52) U.S. Cl. 623/6.12

(57) ABSTRACT

A surgical tool for manipulating a haptic of an intraocular lens. The surgical tool includes an elongated center rail having a traveler slideably disposed within the center rail, the center rail being at least partially disposed in a first housing, an actuator operatively connected to the traveler and fixedly connected to the center rail, a conduit partially disposed within the first housing and slideably coupled to the traveler, and an engaging member selectively extendable out of the conduit, wherein actuation of the actuator manipulates the engaging member.

### CROSS-REFERENCE TO RELATED APPLICATION

This application claims priority to U.S. Provisional Application No. 61/253,636, filed on Oct. 21, 2009, the entirety of which is hereby incorporated by reference.

### BACKGROUND OF THE INVENTION

1. Field of the Invention

This invention relates to a surgical tool for implanting an intraocular lens into a human eye. Particularly, this invention relates to a surgical tool capable of grasping and orienting at least a haptic of an intraocular lens.

2. Description of Related Art

The field of refractive surgery has evolved rapidly during the past few decades. Current surgical devices used by refractive surgeons, however, are not particularly tailored for individual surgeries. Specifically, the most commonly performed refractive surgical procedures, such as, for example, cataract extraction with intraocular lens implantation, do not have tools or equipment specifically configured to effectively and efficiently facilitate the procedure. In particular, surgeons generally adapt surgical tools designed for other types or kind of surgeries when implanting and orienting intraocular lenses having haptics. Because such surgical devices are not specifically configured for implanting intraocular lenses having haptics, the tools do not afford the surgeons with a manner of precisely and safely manipulating the intraocular lens.

For example, in some instances, surgeons have been known to use tools that designed for bisecting lenses wherein such tools were normally used to remove a lens from the eye. However, using such a tool to position the intraocular lens would run a significant risk of severing the portion of the lens being manipulated if the tool was fully actuated. Thus, there is a need in the art for a surgical tool capable of gripping a haptic of an intraocular lens without severing the lens. The disclosure fills the need in the art by providing such a surgical tool.

### SUMMARY OF THE INVENTION

It is an aspect of this invention to provide a surgical tool for manipulating a haptic of an intraocular lens without severing the lens.

In an aspect of the present invention, the surgical tool includes an elongated center rail having a traveler slideably disposed within the center rail, the center rail being at least partially disposed in a first housing, an actuator operatively connected to the traveler and fixedly connected to the center rail, a conduit partially disposed within the first housing and slideably coupled to the traveler, and an engaging member selectively extendable out of the conduit, wherein actuation of the actuator manipulates the engaging member.

### BRIEF DESCRIPTION OF DRAWINGS

Various aspects of the present invention are illustrated by way of example, and not by way of limitation, in the accompanying drawings, wherein:

FIG. 1 illustrates a perspective view of a capsule capture snare in a non-operative position, according to a first embodiment of the present invention;

FIG. 2 illustrates a perspective view of a capsule capture snare in a non-operate position, according to a second embodiment of the present invention;

FIG. 3 illustrates a perspective view of a capsule capture snare in a non-operative position, according to a third embodiment of the present invention;

FIG. 4 illustrates a side view of the capsule capture snare illustrated in FIG. 1 in a non-operative position;

FIG. 5 illustrates a top view of the capsule capture snare illustrated in FIG. 1 in a non-operative position;

FIG. 6 illustrates a top view of the capsule capture snare illustrated in FIG. 1 in a partially operative position;

FIG. 7 illustrates a top view of the capsule capture snare illustrated in FIG. 1 in a completely operative position;

FIG. 8 illustrates a rear view of the capsule capture snare;

FIG. 9 illustrates a front view of the capsule capture snare;

FIG. 10 illustrates a partial sectional side view of the capsule capture snare illustrated in FIG. 1 in a non-operative position;

FIG. 11 illustrates a partial sectional side view of the capsule capture snare illustrated in FIG. 1 in a fully operative position;

FIG. 12 illustrates a partial view of the set screw feature, wherein a wire is secured by a knot or ball;

FIG. 13 illustrates a partial view of the set screw feature, wherein a wire is secured by a groove;

FIG. 14 illustrates a partial sectional side view of the capsule capture snare illustrated in FIG. 2 in a non-operative position, wherein a wire is secured by a set screw located on a center rail;

FIG. 15 illustrates a partial sectional side view of the capsule capture snare illustrated in FIG. 2 in a fully operative position;

FIG. 16 illustrates a partial sectional side view of the capsule capture snare illustrated in FIG. 3 in a non-operative position, wherein a wire is secured by a slot located on a center rail;

FIG. 17 illustrates a partial sectional side view of the capsule capture snare illustrated in FIG. 3 in a fully operative position; and

FIG. 18 illustrates a partial view of a slot located on a center rail having a hinge to secure a wire.

### DETAILED DESCRIPTION

The detailed description set forth below in connection with the appended drawings is intended as a description of various embodiments of the present invention and is not intended to represent the only embodiments in which the present invention may be practiced. The detailed description includes specific details for the purpose of providing a thorough understanding of the present invention. However, it will be apparent to those skilled in the art that the present invention may be practiced without these specific details.

The capsule capture snare (10) may be primarily used in the field of cataract surgery and more specifically for the manipulation of an intraocular lens. Typically, during cataract surgery, a patient's crystalline lens, which is clouded, is removed to allow light to pass to the retina. The crystalline lens is replaced by an intraocular lens assembly that is injected into the space defined inside the lens capsule after the cataract has been removed. The lens must then be properly oriented by the surgeon. Such a procedure requires precise manipulation of the intraocular lens assembly. When performing the surgery with an intraocular lens assembly having a base lens with two haptic elements, it is desirable for the surgeon to be able to isolate one of the haptics outside the capsule and leave the remaining haptic inside the capsule. In other words, it is preferable that the surgeon be able to precisely orient the lens so that the front haptic sits or is positioned in front of the lens capsule. In the desired configuration, the base lens of the assembly is located in the capsule and the haptics are located in front of the capsule. The present invention achieves these desirable results by providing a capsule capture snare (10) that allows the surgeon to easily ensnare the haptic element and precisely reposition the components of the intraocular lens assembly.

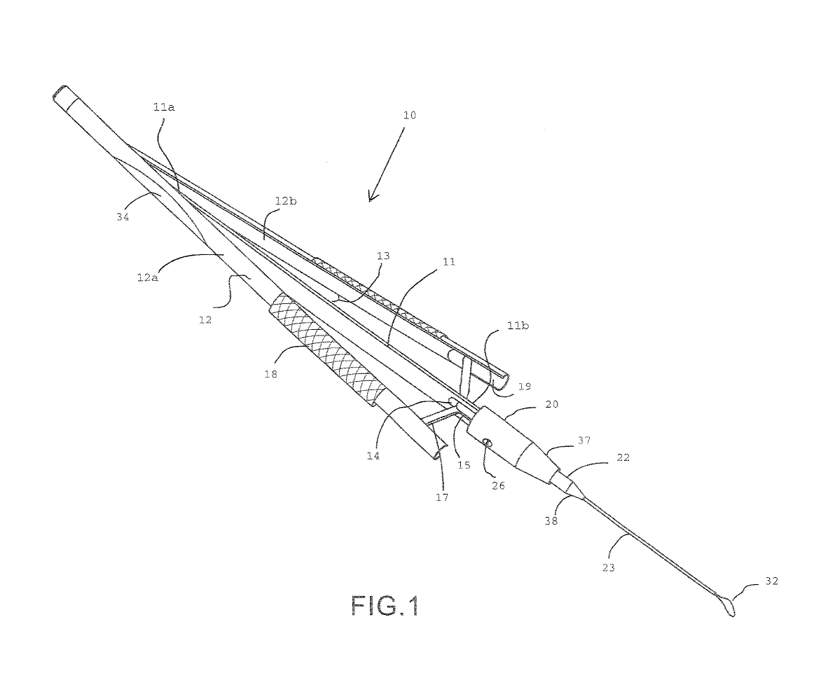

FIG. 1 illustrates an exemplary embodiment of the present invention. In FIG. 1, the capsule capture snare (10) is shown in a non-operative state. The non-operative state occurs when no external force is being applied to the capsule capture snare (10). The non-operative state is described in detail below. The capsule capture snare (10) includes a center rail (11) having a distal end (11a) and a proximal end (11b). The center rail (11) may be composed of any material, including known or later developed plastics and metals, that are suitable for medical, and in particular, surgical procedures. In FIG. 1, the center rail (11) is illustrated as a rod having a rectangular shape. However, the center rail (11) may be formed as any suitable geometric shape such as, for example, a cylinder, so long as the center rail (11) is capable of receiving and operating with the structural elements described below.

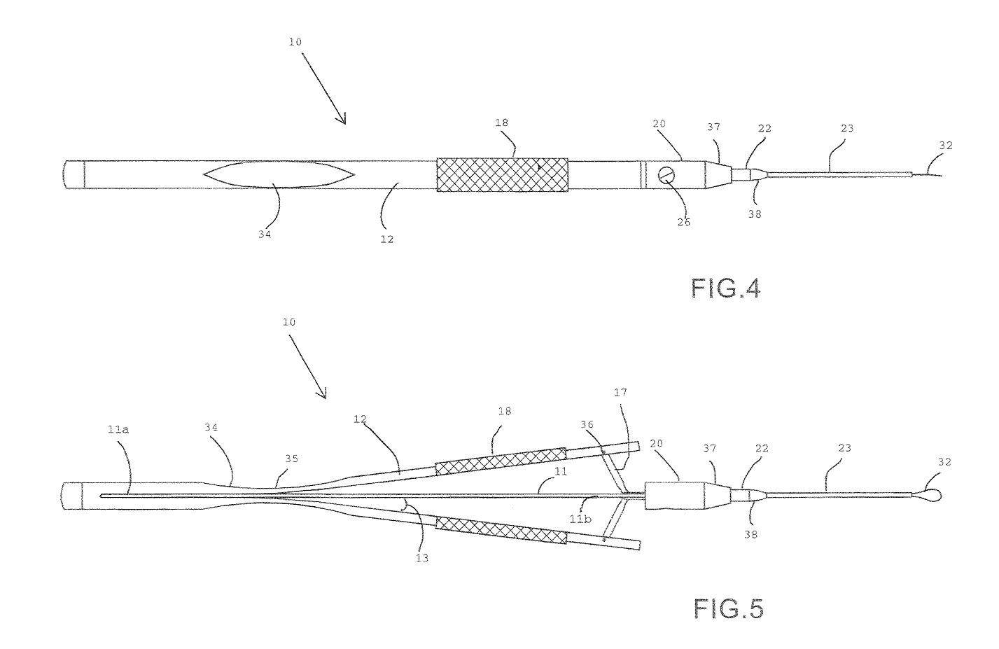

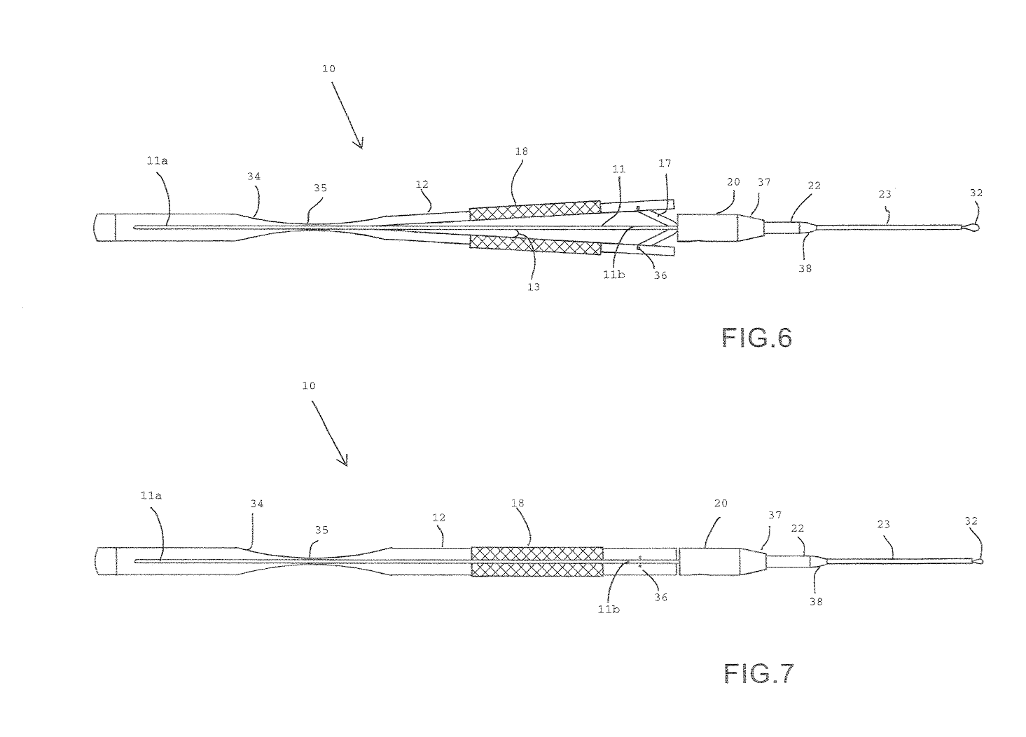

An actuator (12) engages the center rail (11) at the distal end (11a) of the center rail (11). As illustrated in FIG. 1, in an exemplary aspect, the actuator may be a pair of actuating arms (12). The actuating arms (12) extend from the distal end (11a) of the center rail (11) towards the proximal end (11b) of the center rail (11). The actuating arms (12) may be manufactured from any suitable material, including known or later developed plastics and metals. Preferably, the actuating arms (12) may be made of a material that allows for easy gripping by the human hand and that is also safe to use for medical purposes. In accordance with the medical use of the device, the actuating arms (12), as well as the entire capsule capture snare (10), may be made of a material that is easily sterilized and/or disposable. For example stainless steel, titanium or any suitable sterilizable metal may be used. Plastics such as polyvinyl chloride or any suitable sterilizable and/or disposable plastics may also be used. In FIGS. 1-7, 11, 15, and 17, the actuating arms (12) are illustrated in the shape of axially bisected cylinders having a rounded outer surface (12a) and a planar inner surface (12b). The actuating arms (12) are oriented such that the flat planar surfaces (12b) of the opposing actuating arms (12) face each other. When in the completely operative position, the actuating arms (12) mate with the opposing sides of the center rail (11) to form an essentially elongated cylinder. This feature is best shown in FIG. 7.

At an area near or close to the proximal end of the actuating arms (12), the center rail (11) is engaged by the actuating arms (12) wherein the actuating arms (12) project at an angle (13) away from a longitudinal axis of the center rail (11) when force is not being applied to the actuating arms (12). Any suitable method of securing the actuating arms (12) to the center rail (11) may be used. Preferably, the manner in which the proximal ends of the arms (12) engage the center rail (11) should be chosen such that the actuating arms (12) can be actuated a plurality of times to easily control the projection angle (13). As shown in FIGS. 5-7, the actuating arms (12) can be formed as a single, integral piece.

The actuating arms (12) are biased away from the center rail (11) when in the non-operative state. The operator of the capsule capture snare (10) may overcome the biasing force by squeezing the actuating arms (12) towards the center rail (11). As the operator continues to apply force to the actuating arms (12), the capsule capture snare (10) is manipulated from the non-operative state to a series of partially operated states, and ultimately to a fully operative state. In the non-operative state the engaging member (32), described below, is able to encompass the necessary component of lens assembly. In the partially operated state, the engaging member (32) becomes tighter around the component of the lens assembly. Finally, in the fully operated state the engaging member (32) tightly engages the component of the lens assembly. When the operator reduces the amount of force being applied to the actuating arms (12), the biasing force begins to return the capsule capture snare (10) to the non-operative state. The process involved in operating the device is described in detail below.

The actuating arms (12) are biased away from the center rail (11) through a biasing force imparted by a biasing mechanism. In the exemplary embodiment, the biasing mechanism is comprised by the manner in which the actuating arms (12) are formed or manufactured. As shown in FIG. 5, the actuating arms (12) may be formed as a single, integral piece. The actuating arms (12), when formed as single piece, may be manufactured in such a way that the actuating arms (12) are predisposed to extend away from each other in a non-operative state. The distal end of the single piece may surround the distal end of the center rail (11a). Therefore, when the distal end of the center rail (11) is disposed between the actuating arms (12), the actuating arms (12) extend away from the center rail (11) and each other as they move to the predisposed position. However, the biasing mechanism may comprise at least one biasing device disposed between opposing surfaces of the distal end of a first actuating arm (12) and the distal end of a second actuating arm (12) or between opposing surfaces of the distal ends of the actuating arms (12) and the corresponding surfaces of the center rail (11). The biasing device may be any device that imparts a suitable biasing force such as, for example, springs, wedges, fixed arms, adjustable arms, spring loaded telescoping arms, and the like. Furthermore, the biasing device may be a hydraulically or pneumatically actuated piston and cylinder arrangement.

In the exemplary embodiment, as best seen in FIGS. 5-7, each actuating arm (12) further includes a reduced thickness portion (34) relative to the maximum thickness of the actuating arms (12), where the thickness of the actuating arm (12) is greatly reduced. The reduced thickness portion (34) extends in an axial direction and tapers to and away from a midpoint (35) of the reduced thickness portion (34). At the midpoint (35), the thickness of the actuating arm (12) is most greatly reduced. Thus, the reduced thickness portion (34) forms a concavity in the actuating arm (12). As shown in FIGS. 5-7, the reduced thickness portions (34) may be identical in shape and located on the actuating arms (12) such that they are symmetrically opposite each other. The reduced thickness portions (34) may serve the function of allowing the heel of a thumb to comfortably rest on the device. The reduced thickness portions (34) may also act as a rest for the operator's hand, thereby allowing the contours of the hand to comfortably fit around the device while preventing slippage during operation. Furthermore, having a reduced thickness portion (34) makes the device lighter.

The actuating arms (12) may further include gripping portions (18). The gripping portions (18) are shown as knurled surfaces, but it is within the scope of the present invention for any suitable non-slip technique and/or material to be used. For example, a non-slip material, such as rubber, may be removeably or permanently placed around the actuation arms (18). The gripping portions (18) allow for the operator of the device to hold and operate the device in the optimal manner. The gripping portions (18) also act as an indicator to the operator as to where to apply pressure to best actuate the device.

The center rail (11) further includes an elongated bore (14) defined therein and located near the proximal end of the center rail (11). A traveler (15) slidingly moves forward and rearward in an axial direction within the bore (14). The traveler (15) may be made of any suitable material that is capable of repeated movement while in constant contact with center rail (11). Preferably, the traveler (15) may be made of stainless steel, plastic, or rubber. The traveler (15) has a shape corresponding to the configuration of the bore (14) enabling the traveler (15) to fit and axially slide within the bore (14).

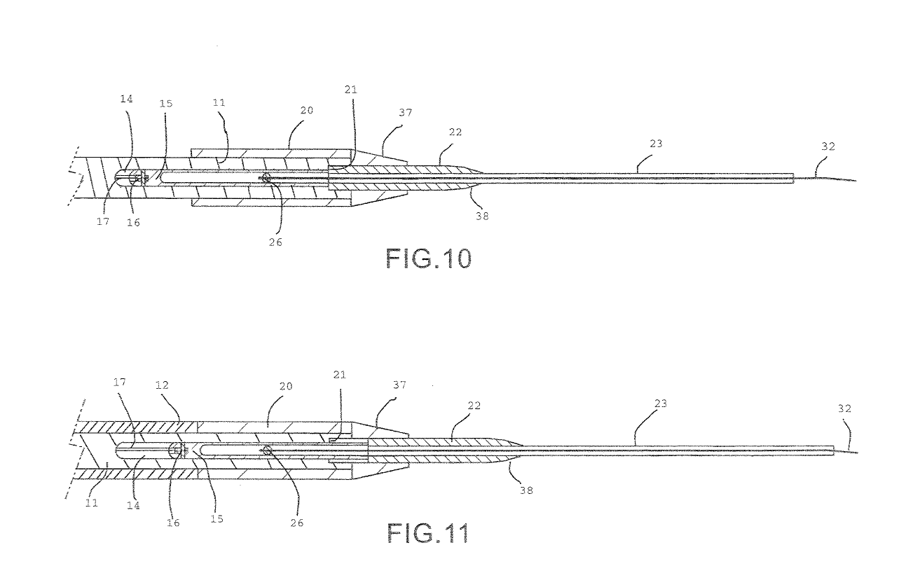

A connecting member (16), best seen in FIGS. 10 and 11, is located at the distal end of the traveler (15). The connecting member (16) is connected to a coupling mechanism (17) on opposing sides of the center rail (11). In an exemplary aspect, as illustrated in FIGS. 1-3, the coupling mechanism (17) may be a pair of secondary arms (17). Both of the secondary arms (17) are attached to the connecting member (16) at an approximately common axial point. The other ends of the secondary arms (17) are connected to actuating arms (12) by a connecter, such as a pin (36). The actuating arms (12) are biased away from the center rail (11) when no force is being applied to the actuating arms (12). When force is applied to the actuating arms (12) to overcome the biasing force, the force is transferred to the secondary arms (17). Because the secondary arms (17) are attached to the traveler (15) by the connecting member (16), the secondary arms (17) begin to push against traveler (15). As the secondary arms (17) push against the traveler (15), the traveler (15) will begin to move axially within the bore (14). Thus, when force is applied to the actuating arms (12), the ends of the secondary arms (17) that are connected to the connecting member (16) move towards the second housing (22) while the other ends move toward the center rail (11), thereby causing the secondary arms (17) to collapse into a cylindrical shape. The pin (36) allows the secondary arms (17) to rotate about the pin (36) as the traveler (15) moves in an axial direction. The actuating arms (12) may include a cut-away portion (19) in which the secondary arms (17) are received as the traveler (15) begins to move axially towards the proximal end of the device.

The proximal end of the center rail (11) is positioned inside of a first housing (20). At the proximal end of the center rail (11), the width of the bore (14) increases and defines a shoulder (21). A second housing (22) partially telescopes in and out of a bore defined in the first housing (20) such that a distal end of the second housing (22) abuts the shoulder (21) when the device is not being operated.

FIG. 9 is a front view showing the second housing (22) within the first housing (20). The inner diameter of the second housing (22) is slightly smaller than a width of the bore (14) at a point where the bore (14) width is largest. While the second housing (22) is small enough to fit within the larger width section of the bore (14), the housing (22) is too large to fit within the smaller width portion of the bore (14). Therefore, the second housing (22) can fit within the first housing (20), but only enough for the distal end to abut against the shoulder (21) while the proximal end remains outside of the housing (20). The second housing (22) further includes a conical portion (37) which tapers in a direction from the distal end of the second housing (22) toward a proximal end of the second housing (22).

As shown in FIGS. 10 and 11, the second housing (22) is fixed to the proximal end of the traveler (15). In FIG. 10, the traveler (15) is in the non-operative position. In this position the second housing (22) abuts against the shoulder (21). In FIG. 11, the traveler (15) is in a completely operated position. In the completely operative or operated position, the second housing disengages from the shoulder (21) and extends away from the shoulder (21) and the first housing (20).

The second housing (22) includes another conical portion (38) which tapers in a direction from the distal end of the second housing (22) toward a proximal end of the second housing (22). An elongated hollow tubular shaped conduit (23) rests inside and is attached to the second housing (22), and extends from the distal end of the second housing to a point outside of the second housing (22). A wire, cable or filament member (24) extends completely through the tubular conduit (23) and may be secured by a securing mechanism (26), for example a screw, located on the first housing (20). The other end of the wire forms an engaging member (32). In an exemplary aspect, the engaging member (32) may be in the shape of, for example, a loop or noose, and emerges from the tubular conduit (23) at the proximal end of the tubular conduit (23). The engaging member (32) fits around the haptic of the intraocular lens assembly, allowing the operator or surgeon to manipulate the location of the lens assembly. While the figures illustrate the engaging member (32) as a wire loop, the engaging member (32) may be formed as any suitable structure that is capable of gripping the haptic. The engaging member (32) may be substituted for with an engaging member configured to securely hold any component of the lens assembly so as to accurately position the intraocular lens within the eye. For example, the engaging member may be tongs, teeth, prongs, hooks, a multi-fingered gripper, or pincers.

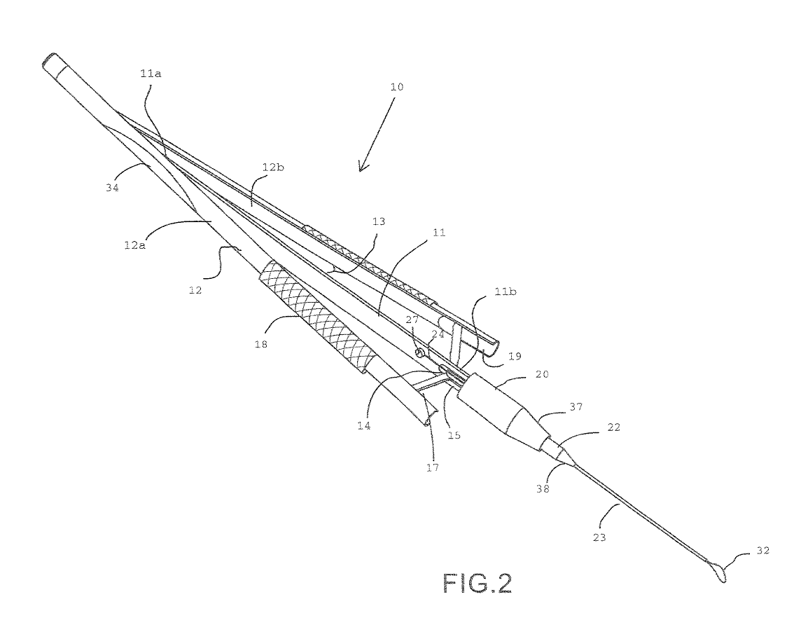

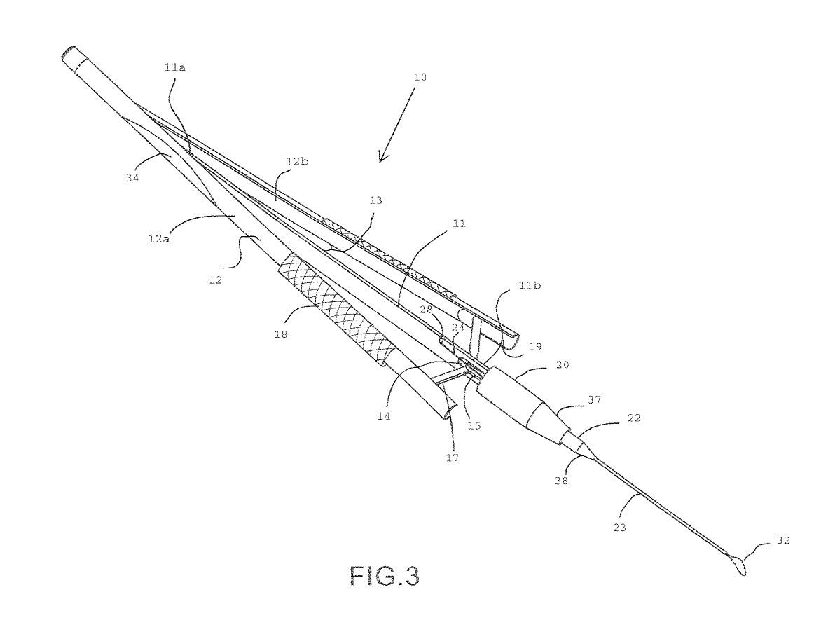

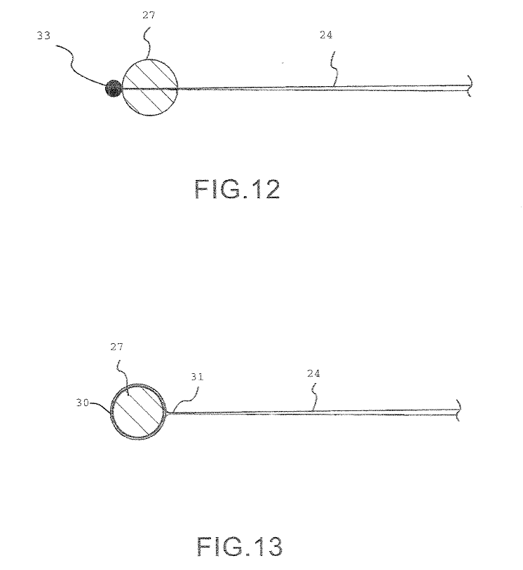

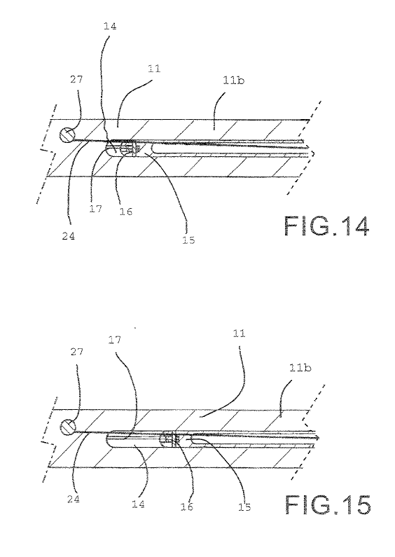

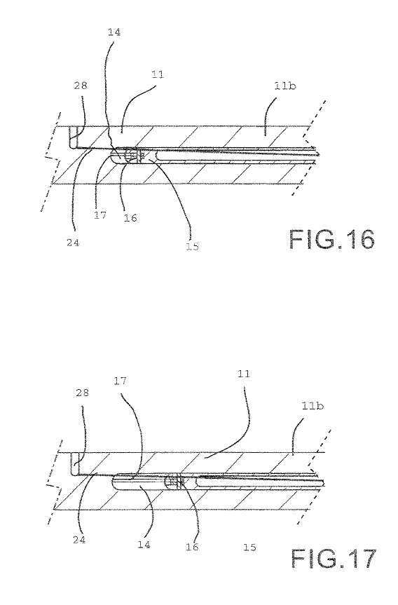

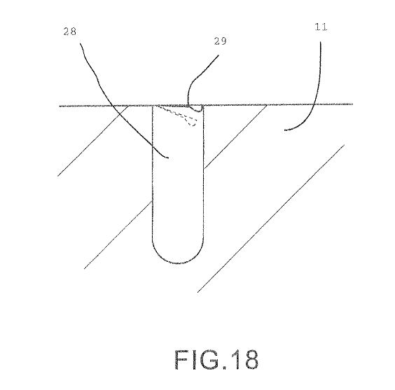

The wire may be secured to the capture capsule snare (10) by any means that holds it in place. For example, FIG. 2 demonstrates how the wire can be secured by a set screw (27) located on the center rail (11). FIGS. 14 and 15 show a side view of the set screw (27) on the center rail (11) when the device is open and closed. FIG. 3 shows an embodiment wherein the wire (24) is secured by means of a slot (28), which can include a living hinge (29). FIGS. 16 and 17 show a side view of the slot (28) when the device is open and closed. FIG. 18 shows an optional hinge (29) for securing the wire (24) in slot (28). FIGS. 12 and 13 show alternative ways to secure the wire using a set screw. In FIG. 12, a knot or ball end (33) is held in place by the set screw. FIG. 13 shows the wire (24) resting in a groove (30) in the set screw, which is secured by a twist (31).

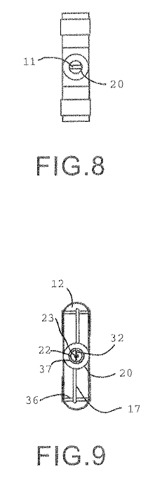

FIG. 8 illustrates a rear view of the capture capsule snare and FIG. 9 shows a front view of the capture capsule snare.

The operation of the device will now be described. FIG. 5 shows the device in the non-operated state. When the device is not yet being operated, the actuating arms (12) are biased away from the center rail (11). During this time, the traveler (15) is in a non-operative or rested position, as shown in FIGS. 10, 14, and 16. Because the second housing (22) is connected to the traveler (15), when the traveler is in the non-operative position, the second housing (22) is in the non-operative or rested position and abuts the shoulder (21). As such, the tubular conduit (23) connected to the second housing (22) is also pulled farther into the capsule capture snare (10). Thus, when the traveler (15) is pulled back into the capsule capture snare (10), more of the wire (24) is able to extend from the tubular conduit (23). With more of the wire (24) free from the elongate tube (24), the engaging member (32) will be in the most open position. For example, when the engaging member (32) is a loop, the diameter of the loop will be largest when the capsule capture snare (10) is fully operated. The engaging member (32) can then be placed around a haptic.

FIGS. 10 and 11 show the movement of the traveler (15) during operation of the device. FIGS. 5, 6, and 7 show the movement of the arms (12, 17) and the resulting change in size of the engaging member (32). When the operator of the device squeezes the actuating arms (12), the force is transferred to the secondary arms (17) are connected to the actuating arms (12). Because the secondary arms (17) are connected to the traveler (15) by the connecting member (16), the force is further transferred to the traveler (15). Furthermore, the traveler (15) is free to move in an axial direction within the bore (14) of the center rail (11). Therefore, as the force is transferred from the actuating arms (12) to the secondary arms (17), and finally to the traveler (15), the traveler begins to move axially towards the proximal end of the center rail (11). Because the second housing (22) is connected to the traveler (15) and the tubular conduit (23) is connected to the second housing (22), as the traveler (15) moves in an axial direction toward the proximal end of the device, so does the second housing (22) and the tubular conduit (23). The more force applied to the actuating arms (12), the more the tubular conduit (23) will ultimately extend from the first housing (20), thereby reducing the amount of wire (24) extending out of the tubular conduit (23). Thus, the more the actuating arms (12) are compressed, the tighter the grip of the engaging member (32) will be. For example, when the engaging member (32) is a loop, the diameter of the loop will become smaller as the actuating arms (12) are compressed. As the engaging member (32) gets smaller, it will tighten around the haptic. Then, the operator may manipulate the haptic in order to precisely position the lens assembly outside of the capsule. After the haptic is precisely positioned, the operator may release the biasing force being applied to the arms (12), allowing the engaging member (32) grip to become looser, thereby releasing the haptic. For example, when the engaging member (32) is a loop, the diameter of the loop will increase as the biasing force being applied to the arms (12) is released. Once the haptic is released, the operator may remove the capsule capture snare (10) from the eye while leaving the intraocular lens in the desired location.

When the force being applied to actuating arms (12) is released, the process described above occurs in reverse. As the applied force is released, the actuating arms (12) begin to move away from the center rail (11), thereby increasing the projection angle (13). Because the secondary arms (17) are connected to the actuating arms (12) by the pin (36), the secondary arms (17) pivot around the pin (36) and expand outwardly away from the longitudinal axis of the center rail (11). The secondary arms (17), being attached to the traveler (15) by connecting member (16), pulls the traveler axially towards the distal end of the center rail (11). As the traveler (15) moves towards the distal end of the center rail, the traveler (15) pulls the second housing (22) in the same direction. The second housing (22) will eventually abut against the shoulder (21) when no force is applied to the actuating arms (12). The second housing (22), being attached to the tubular member (23), pulls the tubular member (23) towards the distal end of the capsule capture snare (10), thereby exposing more of the engaging member (32). Once the force has been removed from the actuating arms (12), the capsule capture snare (10) will ultimately be in the same non-operative state as before it was actuated.

While the preferred embodiment is described above, it is within the scope of the invention to include any suitable means of actuating the capsule capture snare (10). For example, instead of applying force via the actuating arms (12), a trigger mechanism may be implemented wherein pulling the trigger would cause the traveler (15) to move as described above. Alternatively, a button may be used, wherein pressing the button causes the traveler (15) to move. The trigger or button may be used in conjunction with sensors in order to accurately determine the extent of actuation of the capsule capture snare (10). The sensors may be any suitable sensor, for example, electronic, piezoelectric, ultraviolet, or chemical. Furthermore, the traveler (15) may be directly biased by a spring so that after the capsule capture snare (10) is actuated the traveler will return to a non-operative position.

Instead of the actuating arms (12) and secondary arms (17) causing the traveler (15) to move as described above, the motion may also be enacted via a beveled gear arrangement or any other suitable gear arrangement such as a worm drive. The motion may also be enacted via any other suitable means such as hydraulics, pneumatics, springs, a fixed spool, or any combination thereof.

What is claimed is:

1. A surgical tool for manipulating an intraocular lens assembly, the surgical tool comprising:
an elongated center rail having a traveler slideably disposed within the center rail, the center rail being at least partially disposed in a first housing;
an actuator operatively connected to the traveler and fixedly connected to the center rail;
a conduit partially disposed within the first housing and slidably coupled to the traveler; and
an engaging member selectively extendable from the conduit,
wherein actuation of the actuator manipulates the engaging member.

2. The surgical tool of claim 1, further comprising a coupling mechanism operatively connecting the traveler to the actuator.

3. The surgical tool of claim 1, wherein the actuator comprises a pair of first arms having a common connection point.

4. The surgical tool of claim 2, wherein the coupling mechanism is a pair of secondary arms.

5. The surgical tool of claim 2, wherein actuation of the actuator imparts a sliding force on the traveler via the coupling mechanism.

6. The surgical tool of claim 1, wherein actuation of the actuator imparts a sliding motion on the traveler in an axial direction.

7. The surgical tool of claim 1, further comprising a second housing slideably retained in the first housing, wherein the conduit is coupled to and extends out of the second housing.

8. The surgical tool of claim 7, wherein actuation of the actuator imparts a sliding force on the second housing in an axial direction, and wherein a sliding motion of the second housing in the axial direction imparts an axial movement to the conduit.

9. The surgical tool of claim 8, wherein the axial movement of the conduit manipulates the engaging member.

10. The surgical tool of claim 1, wherein the engaging member comprises a wire loop, and wherein a diameter of the loop decreases when the actuator is actuated.

11. The surgical tool of claim 10, wherein the engaging member is secured to the center rail via a set screw.

12. The surgical device of claim 11, wherein the engaging member is secured within a groove defined in the set screw.

13. The surgical tool of claim 7, wherein the second housing is at least partially retained on the center rail and abuts a shoulder of the traveler in a non-operative state.

14. The surgical tool of claim 13, wherein an end of the second housing abuts an end of the traveler.

15. The surgical tool of claim 14, wherein actuation of the actuator imparts a sliding force on the traveler in an axial direction, and wherein a sliding motion of the traveler in the axial direction imparts an axial movement to the second housing.

16. The surgical tool of claim 7, wherein an inner diameter of the first housing and an inner diameter of the second housing decrease in an axial direction.

17. The surgical tool of claim 4, wherein the secondary arms extend substantially transverse relative to a longitudinal axis of the surgical tool in a non-operative state.

18. The surgical tool of claim 1, wherein the actuator is biased in a direction away from the center rail.

19. The surgical tool of claim 3, wherein each arm of the pair of first arms project at an angle from a longitudinal axis of the center rail, and wherein the angle decreases when the actuating arms are actuated.

20. The surgical tool of claim 4, wherein each arm of the pair of secondary arms is pivotally connected to the actuator via a pin.

21. The surgical tool of claim 1, wherein the engaging member is fixed to the first housing.

22. The surgical tool of claim 1, wherein the engaging member is fixed to the center rail.

23. The surgical tool of claim 1, wherein the first housing includes a conical portion which tapers in an axial direction.

24. The surgical tool of claim 7, wherein the second housing includes a conical portion which tapers in an axial direction.

25. The surgical tool of claim 1, wherein the engaging member includes one or more of tongs, teeth, prongs, hooks, fingers, or pincers.

26. The surgical tool of claim 18, wherein a biasing device biases the actuator.

27. The surgical tool of claim 2, wherein the coupling mechanism biases the actuator in a direction away from the center rail.

28. The surgical tool of claim 26, wherein the biasing device is one of a spring, a wedge, a fixed arm, an adjustable arm, a spring loaded telescoping arm, and a hydraulically or pneumatically actuated piston and cylinder.

29. The surgical tool of claim 1, wherein the actuator includes a reduced thickness portion having a smaller thickness than a non-reduced thickness portion.

30. The surgical tool of claim 1, wherein the actuator includes a gripping portion having a non-slip surface.

31. The surgical tool of claim 1, wherein the non-slip surface is knurled.

32. The surgical tool of claim 1, wherein the traveler is disposed within a bore of the center rail.

33. The surgical tool of claim of claim 2, wherein an end of the coupling mechanism is received in a cut-away portion of the actuator.

34. The surgical tool of claim 20, wherein an end of each arm of the pair of secondary arms are received in a cut-away portion of the actuator.

35. The surgical tool of claim 34, wherein the pin passes through the end of each arm of the pair of secondary arms and through the cut-away portion of the actuator.

36. The surgical tool of claim 18, wherein the actuator is manufactured as a single piece and is predisposed to biasing in the direction away from the center rail.
